(19) United States
(12) Patent Application Publication
(10) Pub. No.: US 2015/0082465 A1
(43) Pub. Date: Mar. 19, 2015

(54) System and Method for Whole-Animal High-Throughput Compound Screening

(71) Applicant: System959, LLC, New York, NY (US)

(72) Inventors: Saul Kato, New York, NY (US); Jennifer Garrison, San Francisco, CA (US)

(21) Appl. No.: 14/485,619

(22) Filed: Sep. 12, 2014

Related U.S. Application Data
(60) Provisional application No. 61/877,501, filed on Sep. 13, 2013.

(51) Int. Cl. A61K 49/00; G01N 21/64

(52) U.S. Cl. CPC A61K 49/0008; G01N 21/6486; G01N 2333/43534; G01N 2500/04
USPC 800/3; 424/9.2; 506/10

(57) ABSTRACT

The present invention provides systems and methods for performing assays for determining the presence of one or more compounds or analytes in a sample using whole-animals. The systems and methods are particularly suited to high-throughput screening techniques to identify compounds that are effective in a whole animal based system. The methods of the invention have broad application in high-throughput drug discovery and identification, particularly for molecules which are associated with disease and disease progression.

### CROSS REFERENCE TO RELATED APPLICATIONS

This application claims priority under 35 USC §119(e) to U.S. Provisional Patent Application No. 61/877,501, filed on Sep. 13, 2013, which is herein incorporated by reference in its entirety.

### FIELD OF INVENTION

The present invention relates to microfluidics systems for performing assays using high-throughput screening systems for use in the identification and discovery of compounds that can serve as therapeutics in the treatment and/or prevention of diseases and disorders.

### BACKGROUND

Compounds that modulate the activity of their targets, such as enzymes and receptors, are important therapeutic agents. These compounds can cause a change in the biological status of the cell containing the target, and the change may be therapeutically beneficial to the host of the cell. The discovery and development of new drugs, therefore, tries to identify compounds that modulate the targets.

Tremendous advances have been made in the development of new tools to identify targets and the corresponding chemicals that interact with these targets and phenotypes. One technique for drug screening is high throughput screening (HTS). High Throughput Screening leverages automation to quickly assay the biological or biochemical activity of a large number of active compounds, antibodies, or genes that modulate a particular target or biomolecular pathway. It is useful for discovering ligands for receptors, enzymes, ion-channels or other pharmacological targets, pharmacologically profiling a cellular or biochemical pathway of interest, or testing the toxic effects of compounds. It is also a useful method for discovering compounds that modulate a phenotype of interest, even when the target is not known. The results of these experiments provide starting points for drug design and for understanding a particular biochemical process. Typically, HTS assays are performed in microtiter plates with a 96, 384 or 1536 chamber format.

Aside from in vitro screening, drugs can be screened in cell lines and organisms such as yeast and bacteria, and these techniques have historically played an important role in identifying bioactive molecules. However, the use of these microorganism models for drug discovery is hindered by the fact that unicellular organisms lack major families of drug targets found in metazoans (e.g. tyrosine kinases, nuclear hormone receptors). In addition, many drugs show physiological activity only in multicellular full organisms due to the complex network of interactions within a biological system. Testing on whole animals, such as mice, dogs, and monkeys, can be prohibitively expensive when testing many thousands or millions of compounds, due to the cost and time of rearing, housing, maintaining, and testing animals. Therefore, animal testing is limited to much smaller, more selective compound screens.

It has recently been proposed that a small, fast growing animal, such as Caenorhabditis elegans, could be used for screening compounds [Jean Giacomotto and Laurent Ségalat (2010). High-throughput screening and small animal models, where are we? British Journal of Pharmacology 160, 204-216; Marta Artal-Sanz, Liesbeth de Jong and Nektarios Tavernarakis (2006). Caenorhabditis elegans: A versatile platform for drug discovery. Biotechnology Journal 1, 1405-1418]. A microfluidic device consisting of flow and control layers made from flexible polymers, where the flow layers contain microchannels for manipulating C. elegans, microchambers for incubating C. elegans, immobilizing them for imaging, and delivering media and reagents is discussed in Christopher B. Rohde, et al. (2007) Microfluidic system for on-chip high-throughput whole-animal sorting and screening at subcellular resolution. Proceedings of the National Academy of Sciences of the United States of America 104(35), 13891-13895. Animals in the flow lines can be imaged through a transparent glass substrate using low or high-resolution microscopy.

However, the execution of such an approach has been hampered by several practical difficulties. One significant practical difficulty is that maintaining, breeding, and testing organisms require a large degree of manual handling by lab technicians. Another significant practical difficulty is that C. elegans possesses a thick cuticle and intestinal lining which presents a physical barrier to many chemical species, as well as a large number of xenobiotic pumps and drug detoxification enzymes that prevent or slow the absorption of many compounds. For this reason, seminal studies performing chemical screens in C. elegans have found that worm permeability (bioavailability) of the small molecule can be a more important factor than its potency in determining which compounds score as hits (Andrew R. Burns, kin M. Wallace, Jan Wildenhain, Mike Tyers, Guri Giaever, Gary D. Bader, Corey Nislow, Sean R. Cutler, and Peter J. Roy (2010). A predictive model for drug bioaccumulation and bioactivity in Caenorhabditis elegans. Nature Chemical Biology 6, 549-557). We describe a system and method to overcome these difficulties.

### SUMMARY

The present invention provides methods and microfluidics systems for performing high-throughput screening assays using whole animals for identifying compounds that modulate a target or phenotype of interest. The methods of the invention can enable improved experiments to be performed in the microfluidics modules of the invention, and more accurate and reliable results to be achieved at lower cost and higher speed.

The present invention provides a robotic microfluidic device for maintaining and manipulating C. elegans, delivering media and library of compounds to the animals, and using high-throughput whole-animal sorting and screening to identify compounds.

In one aspect of the invention, an animal-based screening method to identify compounds is provided where an animal is placed within a chamber in a microfluidics device module, contacting the animal with a test compound, and imaging the contents of the chamber in order to obtain results. The method can be a high-throughput screening method, where the animal is a whole animal, an embryo or a larvae. The animal can be wild-type or transgenic C. elegans wherein the transgenic C. elegans comprises enhanced permeability to compounds.

These and other aspects of the present invention will become evident upon reference to the following detailed description. In addition, various references are set forth herein which describe in more detail certain procedures or compositions, and are therefore incorporated by reference in their entirety.

### BRIEF DESCRIPTION OF DRAWINGS

FIG. 1 illustrates a microfluidics module of the invention.

FIG. 2 illustrates a microfluidics platform of the invention for maintaining, handling, and testing of organisms.

FIG. 3 provides representative data from fluorescent dye uptake assay. Wild-type worms do not accumulate gut esterase-activated dye in their tissues, while compound permeable strains do.

FIG. 4 provides representative data from growth assay in presence of drugs. Nemadipine (center boxes) is known to inhibit the growth of C. elegans by antagonizing egl-19, an L-type calcium channel. Verapamil (right boxes), a drug similar to nemadipine, also antagonizes egl-19 but is excluded from wild-type worms.

### DETAILED DESCRIPTION

### I. Definitions

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise.

All publications, patents and patent applications cited herein, whether supra or infra, are hereby incorporated by reference in their entirety.

As used herein, the term “subject” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender.

The terms “specifically binds to” or “specifically immunoreactive with” refers to a binding reaction which is determinative of the presence of the target analyte in the presence of a heterogeneous population of proteins and other biologics. Thus, under designated assay conditions, the specified binding moieties bind preferentially to a particular target analyte and do not bind in a significant amount to other components present in a test sample. Specific binding to a target analyte under such conditions may require a binding moiety that is selected for its specificity for a particular target analyte. A variety of immunoassay formats may be used to select antibodies specifically immunoreactive with a particular antigen. For example, solid-phase ELISA immunoassays are routinely used to select monoclonal antibodies specifically immunoreactive with an analyte. See Harlow and Lane (1988) Antibodies, A Laboratory Manual, Cold Spring Harbor Publications, New York, for a description of immunoassay formats and conditions that can be used to determine specific immunoreactivity. Typically a specific or selective reaction will provide a signal to noise ratio at least twice background and more typically more than 10 to 100 times background.

As used herein, the terms “label” and “detectable label” refer to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, strepavidin or haptens) and the like.

As used herein, a “solid support” refers to a solid surface such as a plastic plate, magnetic bead, latex bead, microtiter plate well, glass plate, nylon, agarose, acrylamide, and the like.

“Specific” in reference to the binding of two molecules or a molecule and a complex of molecules refers to the specific recognition of one for the other and the formation of a stable complex as compared to substantially less recognition of other molecules and the lack of formation of stable complexes with such other molecules. Exemplary of specific binding are antibody-antigen interactions, enzyme-substrate interactions, polynucleotide hybridizations and/or formation of duplexes, cellular receptor-ligand interactions, and so forth.

### II. Overview

The invention pertains to systems and methods for performing assays for determining the biological activity and pharmacological properties of one or more compounds, individually or in combination, using whole-animals. The systems and methods are particularly suited to high-throughput screening techniques to identify compounds that are effective in a whole animal based system.

The invention provides an automated, robotic microfluidics system for maintaining a single or multiple C. elegans through part or all of the full life cycle from egg to death, as well as multiple generations of animals under active selection, with minimal or no manual intervention. In this system, organisms such as C. elegans can be moved, sorted, immobilized, exposed to compounds, and imaged by microscopy for anatomical study, behavioral study, and by using various genetically encoded fluorescent or luminescent reporters, the concentration of various molecular species, such as calcium, the electrical potential, or other characteristics of cells can be monitored in the live animal. Physiologic parameters including but not limited to reproduction, genetic and epigenetic modifications, ingestion, consumption of various materials, and metabolites can be monitored by coupling the system with various micro-titration and analytic techniques.

The invention provides in vivo screening methods to detect and identify substances that affect cell viability, and/or prevent disease or phenotype progression, and/or confer protective effects. The screening methods utilize recombinant C. elegans expressing a detectable marker in sub-groups of cells that display quantifiable phenotypes in the genetic backgrounds of the library of C. elegans permeability mutant strains.

The invention discloses an automated microfluidic platform for maintaining, handling, and testing organisms with a library of synthetic mutants with a variety of permeability characteristics and compound accessibility. The system of the invention enables rapid and efficient high-throughput compound screening on intact metazoan organisms.

### III. Microfluidic System

Microfluidic devices have gained acceptance recently and have significantly influenced the design and the implementation of modern bioanalytical systems. These devices can handle and manipulate small fluid samples in a much more efficient way with the potential of faster assay response times, the simplification of analysis procedures, and smaller samples required for analysis. Microfluidic devices are finding wide applications ranging from synthesis to separations to analysis in applications such as immunoassays, lab-on-a-chip, rapid nucleotide sequencing, and high throughput screening.

Accordingly, an exemplary microfluidic device typically comprises structural or functional features dimensioned on the order of a millimeter-scale or less, which are capable of manipulating small amounts of fluids. Typically, such features include, but are not limited to channels, fluid reservoirs, reaction chambers, mixing chambers, and separation regions. In some examples, the channels include at least one cross-sectional dimension that is in a range of from about 0.1 μm to about 500 μm. The use of dimensions on this order allows the incorporation of a greater number of channels in a smaller area, and utilizes smaller volumes of fluids.

A microfluidic device can exist alone or can be a part of a microfluidic system which, for example and without limitation, can include: pumps for introducing fluids, e.g., samples, reagents, buffers, drugs, and the like, into the system and/or through the system; detection equipment or systems; data storage systems; and control systems for controlling fluid transport and/or direction within the device, monitoring and controlling environmental conditions to which fluids in the device are subjected, e.g., temperature, current, and the like.

In one aspect of the invention, described herein is a microfluidics system having a microfluidics device module having one or a plurality of sealed chambers, and wherein the chamber is in fluid communication with an inlet microfluidic channel and an outlet microfluidic channel. In this way, a fluid can be pumped through the chambers in order to introduce nutrients or to remove biological waste that may accumulate over time. In addition, drugs or nutrients can be pumped into the chambers via the microfluidic channels for use in the culture and study of adults, embryos, and larvae of multi-cellular organisms, organs, tissues, cells or cell lines that can be placed and grown in the chambers. In one aspect of the invention, a plurality of the microfluidics device module, each having one or more chambers, can be connected together within a larger frame to make a multi-purpose microfluidic environment.

In another aspect of the invention, the microfluidics system described herein can be used to perform experiments on whole organisms, such as, for example, C. elegans, fruit fly larvae or embryos (Drosophila melanogaster), zebrafish larvae or embryos (Danio rerio), and other small metazoan organisms placed in the chambers. The changes in the whole animal or the development of the embryos/larvae over a period of days can be monitored for example with or without exposure to drugs or other compounds. Thus, for example, the transparent or partially transparent organisms, such as C. elegans, can be used for imaging of cellular activity or internal process with or without exposure to drugs or other compounds. In other embodiments, experiments can be performed on a monolayer of cells, on a membrane, matrix or other material within the chamber.

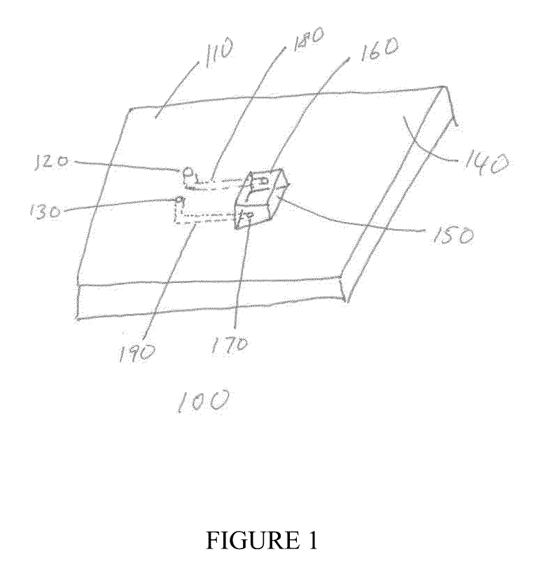

According to first aspect of the invention illustrated in FIG. 1, there is provided a microfluidics device module 100 which comprises a substrate 110. The substrate 110 can be rectangular, circular, oval, or any shape. The substrate 110 can be made from a suitable material that is selected due to its properties, such as good thermal conductivity, surface properties that allow for uniform coating and stability of reagent, and neutrality to the liquid medium to prevent interference with the assay. For this purpose, suitable material for the solid support include flexible polymer such as polydimethylsiloxane, harder polymers, glass, metal, hydrogel, silicon, PETG, polyester, polycarbonate, polyvinyl chloride, polystyrene, SAN, acrylonitrile-butadiene-styrene (ABS), and combination and hybrids thereof, and includes beads, membranes, microtiter wells, strings, plastic, strips, or any surface onto which whole organisms may be deposited or immobilized. Alternatively and equivalently, a commercially available molded solid support can be used in the practice of the invention.

Each microfluidic module can have one or more negative pressure (vacuum) and positive pressure ports to provide the motive force to fluids, or other means of moving the fluids, as well as pressure-conveying ports for driving on-chip valves. In one aspect of the invention, the substrate 110 comprises a microfluidic inlet port 120 and a microfluidic outlet port 130 defining openings in the top surface 140 of the substrate 110. The microfluidic inlet port 120 and microfluidic outlet port 130 may be considered as entrances of the microfluidic channels to an external environment, and can be in the top surface 140, the bottom surface, or the sides of the substrate 110.

A chamber 150 extend downwardly from the top surface 140 of the substrate 110. The chamber 150 has a first opening 160 into a side wall of the chamber 150 and a second opening 170 into the opposite side wall of the chamber 150. The first opening 160 can be an inlet opening, and the second opening 170 can be an outlet opening. In this example, the chamber 150 has a square horizontal cross section. In other embodiments, the chamber can have any shape, and can be circular, oval, rectangular, and the like. An inlet microfluidic channel 180 connects the microfluidic inlet port 120 to the inlet opening 160 in the chamber 150. Similarly, an outlet microfluidic channel 190 connects the microfluidic outlet port 130 to the outlet opening 170 in the sidewalls of the chamber 150.

Large animals, including the embryos and larva of zebrafish, can have dimensions on the order of a few millimeters. To create chambers of these dimensions, channels can be milled into a rigid substrate using a milling machine or laser cutter. For regions requiring rounded channels, including sections containing microfluidic valves, a ball mill with a round tip can be used.

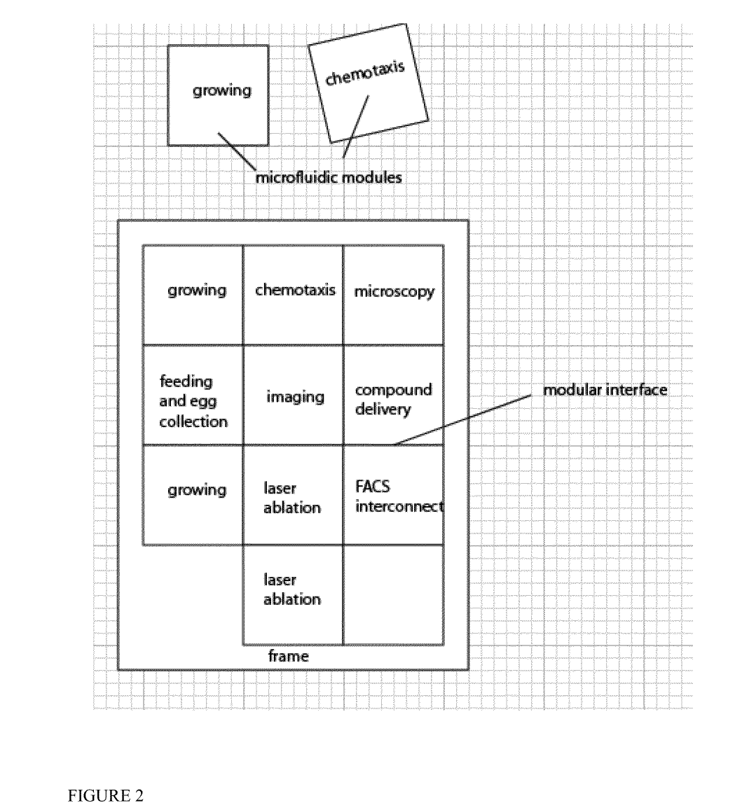

In the embodiment shown in FIG. 1, a microfluidics device module 100 with a single chamber 150 is shown. It will be appreciated that in practice, a microfluidics device module 100 may comprise 32 chambers, 96 chambers, 869 chambers, or any number of chambers that are required. In another aspect of the invention, the microfluidics device consists of a set of microfluidic device modules, typically consisting of fabricated single or multi-layer polymer chips, with a common interface of physical interconnections, allowing any two modules to be connected to each other, as shown in FIG. 2. Multiple chips may be connected together within a larger frame to make a multi-purpose microfluidic environment. The frame may be planar, allowing square or rectangular device form factors or it may be three dimensional, allowing rectangular prism or cubic form factors. The physical interface between modules may consist simply of adjacent faces, or it may consist of male/female sockets akin to a electronic circuit board chip socket, tongue-and-grooves, or other interlocking geometries or fastening methods.

By coordinated switching of on-chip valves, animals can be moved around to different portions of a device as well as between connected devices. Additionally, by coordinated switching of on-chip or off-chip valves, fluids may be routed or from animals for the purposes of test compound or reagent delivery, as well as collection of fluids, tissue, and metabolites for analysis.

In one aspect of the invention, specified microfluidic device modules can be designed to accommodate geometric alignment or physical connection with third party devices such as microscope mounting stages or objectives for the purposes of imaging. Microfluidic modules can be designed to interface with other third party devices such as complex object sorters such as COPAS™ (Complex Object Parametric Analyzer and Sorter, Union Biometrica), cytometers such as FACS (fluorescence activated cell sorter), lasers, irradiators, and other detection system can have similar microfluidic modules for interfacing with the system.

An example of a detection system for automated detection for use with the present invention and associated methods comprises an excitation source, a monochromator (or any device capable of spectrally resolving light components, or a set of narrow band filters) and a detector array. The excitation source can comprise infrared, blue or UV wavelengths and the excitation wavelength can be shorter than the emission wavelength(s) to be detected. The detection system may be: a broadband UV light source, such as a deuterium lamp with a filter in front; the output of a white light source such as a xenon lamp or a deuterium lamp after passing through a monochromator to extract out the desired wavelengths; or any of a number of continuous wave (cw) gas lasers, including but not limited to any of the Argon Ion laser lines (457, 488, 514, etc. nm) or a HeCd laser; solid-state diode lasers in the blue such as GaN and GaAs (doubled) based lasers or the doubled or tripled output of YAG or YLF based lasers; or any of the pulsed lasers with output in the blue.

The emitted light from the organism, the sample or the reactants in the chamber can be detected with a device that provides spectral information for the substrate, e.g., a grating spectrometer, prism spectrometer, imaging spectrometer, or the like, or use of interference (bandpass) filters. Using a two-dimensional area imager such as a CCD camera, many objects may be imaged simultaneously. Spectral information can be generated by collecting more than one image via different bandpass, longpass, or shortpass filters (interference filters, or electronically tunable filters are appropriate). More than one imager may be used to gather data simultaneously through dedicated filters, or the filter may be changed in front of a single imager. Imaging based systems, like the Biometric Imaging system, scan a surface to find fluorescent signals.

### IV. Whole Organism

In one aspect of the invention, the microfluidics system described herein can be used to perform experiments on whole organisms, such as, for example, Caenorhabditis elegans (C. elegans), fruit fly larvae or embryos (Drosophila melanogaster), zebrafish larvae or embryos (Danio rerio), and other small metazoan organisms, or single cell organisms, such as, for example, Saccharomyces cerevisiae (yeast), and other fungi or protozoan organisms, placed in the chambers. The present disclosure will focus on C. elegans, but it should be noted that wherever C. elegans is mentioned, other small animals can be used.

C. elegans is a preferred animal because it has a small diameter, can be micro-manipulated inside microfluidic chips and can be directly exposed to harsh ambient environments. This animal can survive a wide range of environmental stress, temperature ranges, pH conditions, and salinity, and can be kept alive for months without feeding. C. elegans is a non-parasitic soil-living nematode and these animals have genes with vertebrate homologs. The worm's hermaphroditic nature and lifespan of three weeks allows for extensive observations. The C. elegans genome has been completely sequenced and mutants are readily available.

C. elegans can be grown at a relatively low cost because of its easy gene manipulation and small size. C. elegans is suitable for experimental uses because it takes only approximately 3 days to become an adult through four stages, L1, L2, L3, and L4, after hatching from its egg. C. elegans has a simple structure, with only 959 cells in the hermaphrodite excluding reproductive cells, and is transparent. For these reasons, it is easy to directly observe the inside of C. elegans through a microscope. In addition, a cell lineage to an adult from an embryo has been completely identified. It is known from the genome project that the genome of C. elegans is three times that of yeast, and ⅓ to ⅕ that of a human. The genome of C. elegans is 40% similar to that of a human, and shares 75% of the 5,000 human disease genes that are known thus far. For example, homogenous genes for genes important to the metabolism of mammals such as genes for an insulin signaling pathway, genes for an mTOR signaling pathway, and genes for Tubby, SREBP, PPAR, BAR (bile acid receptor), etc. are present in C. elegans. Thus, it can serve as a good model system for the study of human diseases.

In some embodiments, one or more of the nematodes can be C. elegans. In these embodiments, 1, 2, 3, 4, 5, 6, 7, 8, 9, or 10 or more of the chambers have a plurality of nematodes which comprise a plurality of C. elegans. For example, the plurality of nematodes can comprise a plurality of wild type or genetically engineered or transgenic C. elegans. In another example, the plurality of nematodes can comprise a plurality of genetically engineered or transgenic C. elegans having a transgene which is a promoter-reporter construct wherein the reporter encodes a fluorescent or luminescent protein and wherein the promoter is a promoter of a C. elegans gene induced in response exposure to a toxin or stress. The strains of C. elegans for use as nematodes can be any strain of C. elegans, including those described herein. Other strains include, but are not limited to, those that can be acquired from the Caenorhabditis Genetics Center at the University of Minnesota, St. Paul.

In other embodiments, the plurality of nematodes can comprise a plurality of genetically engineered or transgenic C. elegans having a transgene which is a promoter reporter construct wherein the reporter encodes a fluorescent or luminescent protein and wherein the promoter is a promoter of a C. elegans gene induced in response exposure to a toxin or stress. In yet other embodiments, the plurality of nematodes can be one or more, two or more, 3 or more, 4 or more, or 5 or more populations of representative transgenic nematodes (e.g., C. elegans), as described in U.S. patent application Ser. No. 13/476,790 filed May 21, 2012, the entire disclosure of which is incorporated herein by reference.

In order for compounds to reach potential drug targets, they must first physically enter the body of the worm. Wild-type (N2) C. elegans have significant physical barriers, pumps, and detoxification mechanisms that prevent compound accumulation. To overcome this difficulty, we describe a library of genetically modified C. elegans strains that have unique combinations of mutations that confer enhanced small molecule permeability for use in high throughput screening with an automated, robotic microfluidics system. Each C. elegans strain in this library may contain mutations in single or multiple genes that induce full or partial gain or loss of function.

In one aspect of the invention, C. elegans mutant strains that display enhanced permeability to compounds, such as proteins, peptides, and small molecules, but are otherwise phenotypically normal are identified. The mutant C. elegans strains can be produced by known methods, such as those disclosed in Sambrook, et al., Molecular Cloning: A Laboratory Manual (2nd Edition, 1989); Methods In Enzymology (S. Colowick and N. Kaplan eds., Academic Press, Inc.). In one aspect, the present invention is directed to methods for mutating a single gene or multiple genes (e.g., two or more) in C. elegans to obtain mutant strains that display enhanced permeability to compounds. The present invention contemplates several methods for creating mutations in C. elegans. In one embodiment the mutation is an insertion mutation. In another embodiment the mutation is a deletion mutation. In another embodiment the method of mutation is the introduction of a cassette or gene trap by recombination. In another embodiment a small nucleic acid sequence change is created by mutagenesis, such as through the creation of frame shifts, stop mutations, substitution mutations, small insertions mutations, small deletion mutations, and the like.

The invention also is directed to insertional mutagens for making the mutant C. elegans. The invention also is directed to methods in which one or more mutated genes is tagged by a tag provided by the insertional mutagen to allow the detection, selection, isolation, and manipulation of a mutant C. elegans with a genome tagged by the insertional mutagen and allows the identification and isolation of the mutated gene(s). The invention provides methods for making multiple mutations, such as mutations in two or more genes that produce a phenotype cumulatively, C. elegans and tagging at least one of the mutated genes such that the mutation can be rapidly identified.

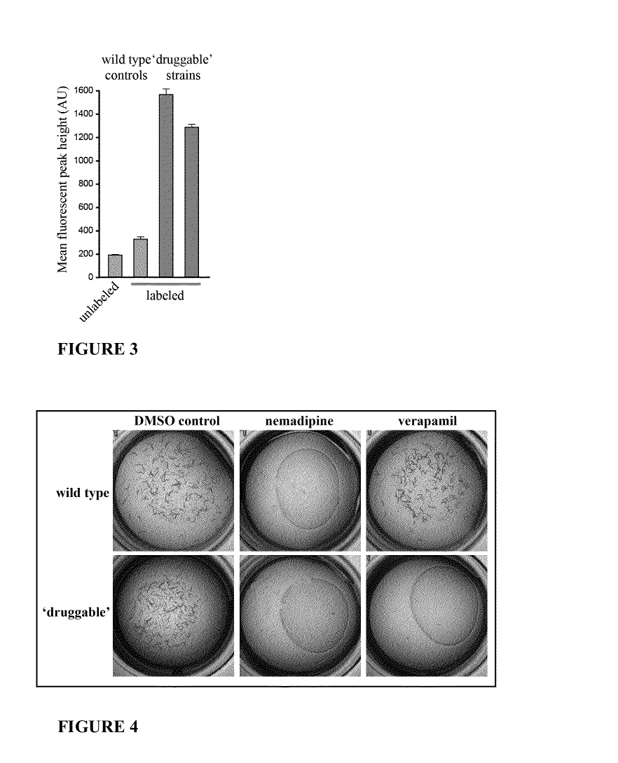

In one aspect of the invention, C. elegans mutant strains that display enhanced permeability to compounds can be identified by in parallel an unbiased forward EMS mutagenesis screen and a candidate-based screen with mutants in genes that may confer permeability, including but not limited to multi-drug resistance proteins, P-glycoproteins, ABC transport proteins, glycosyltransferases, nuclear hormone receptors, organic anion transporters, and fatty acid transporters. The C. elegans mutant strains can be screened for permeability mutants using a combination of fluorescence/luminescence assays, and accessibility assays for compounds that are known to be excluded from wild-type worms (including but not limited to chloroquine, colchicine, nicotine, verapamil and other calcium channel antagonists, heavy metals, ivermectin). Single mutations can be combined combinatorially, and the resultant strains re-screened for health and compound uptake in an iterative fashion until the desired set of mutations have been uncovered. Heath assessments include but are not limited to motility, reproduction, propidium iodide uptake, lifespan, and behavior assays. The cell and tissue small molecule accessibility in the C. elegans mutant strains can be assessed by expressing luciferases in a tissue-specific manner then feeding worms its luciferin cofactor and screening for luminescence. The invention thus provides a panel of druggable mutant strains vary with respect to levels of cell and tissue accessibility. FIG. 3 shows representative data from fluorescent dye uptake assay. Wild-type worms do not accumulate gut esterase-activated dye in their tissues, while compound permeable strains do. Thus, the data shows that mutant strains of C. elegans were produced that display enhanced permeability to compounds.

These strains may allow exogenous compounds that would normally be excluded to enter the worm's body through increased intestinal or cuticle permeability, increased intestinal transport of ingested molecules, increased pharyngeal pumping, decreased function of detoxification enzymes or pumps, a combination of these mechanisms, or by an unknown mechanism. This library may contain strains with permeability that varies by tissue or that varies depending on the chemotype of the exogenous compound. These strains display increased compound permeability with few background phenotypes. Increased permeability is assessed in different tissues and cell types by fluorescence and luminescence assays and accessibility of compounds that are known to be excluded from wild-type (N2) C. elegans worms as determined by phenotypic screening. C. elegans strains in this library may also be engineered to contain constructs for tissue specific, location specific, and/or developmental stage specific expression of endogenous or exogenous human proteins, which may be comprised in plasmids or introduced directly into C. elegans by genome editing. These transgenic C. elegans strains may contain single or multiple copies of transgenes expressing human proteins from extrachoromasomal arrays or integrated into the genome.

Provided are methods for high throughput chemical screens on whole animals with an automated, robotic microfluidics system for substances that modulate complex human disorders which involve multiple tissues or communication between cells and/or tissues and for which underlying causes may not be known in the library of C. elegans permeability mutant strains. One example of such a complex disorder is the disruption of protein homeostasis, which can lead to a number of human diseases. The existence of cell-type and stress specific protein degradation pathways suggests that mechanisms of proteostasis may be tissue-dependent, thus it is important to search for potential modulatory compounds in the context of an intact whole animal. We describe in vivo screening methods to detect and identify substances that affect cell viability, and/or prevent disease or phenotype progression, and/or confer protective effects. The screening methods utilize recombinant C. elegans expressing a detectable marker in sub-groups of cells that display quantifiable phenotypes in the genetic backgrounds of the library of C. elegans permeability mutant strains.

The selected animals can be sorted and dispensed using one of the known methods, or the microfluidics system can be used to sort the animals and move them to the appropriate chamber. Preferably, the method selected for sorting/dispensing is capable of distributing C. elegans, Danio rerio embryos or Drosophila melanogaster embryos into the chambers efficiently, and allows individual organisms to be quickly deposited. Further, each organism can be optically analyzed such that only desired organisms with particular predetermined biological characteristics are deposited. This makes it possible to select identically staged organisms, which greatly increases the uniformity of the testing process. Any of the known sorting processing methods and commercially available equipment can be used.

In other embodiments, the level of expression of the biomarker can be determined using any suitable method. For example, the presence or level of the protein can be detected using an antibody or antigen binding fragment thereof, which specifically binds to the protein. In particular embodiments, the antibody or antigen binding fragment thereof is selected from the group consisting of a murine antibody, a human antibody, a humanized antibody, a bispecific antibody, a chimeric antibody, a Fab, Fab′, ScFv, SMIP, affibody, avimer, versabody, nanobody, and a domain antibody, or an antigen binding fragment of any of the foregoing. In particular embodiments, the antibody or antigen binding portion thereof is labeled, for example, with a label selected from the group consisting of a radio-label, a biotin-label, a chromophore-label, a fluorophore-label, and an enzyme-label. In certain embodiments, the level of expression of the biomarker is determined by using a technique selected from the group consisting of an immunoassay, a western blot analysis, a radioassay, fluorimetry, equilibrium dialysis, electrochemiluminescence, ELISA assay, polymerase chain reaction and combinations or sub-combinations thereof. In particular embodiments, the detection is using electrochemiluminescence, chemiluminescence, fluorogenic chemiluminescence, fluorescence polarization, and time-resolved fluorescence.

The transgenic animals may carry a label to facilitate their detection. In some such embodiments, this may be a fluorescent label. Each animal may carry a different fluorescent label. However, the detectable label need not be a fluorescent label but can be any label. One method for detecting the fluorescent label comprises using radiation of a wavelength specific for the label, or the use of other suitable sources of illumination. The fluorescence from the label can be detected by a CCD camera or other suitable detection means.

### V. Robotic Systems

The microfluidic systems and methods provided herein can take advantage of robotic systems and equipment for storing and moving these microfluidic modules as chamber as robotic systems for rapidly dispensing liquids in and out of the plates. By combining an automated microfluidic platform for maintaining, handling, and testing organisms with a library of synthetic mutants with a variety of permeability characteristics and selectivity, this system enables high-throughput compound screening on intact metazoan organisms.

The robotic system includes programmable robotic arm manipulators to install, remove, and configure modules of the microfluidics module array. When snapped into place, the modules make tight junctions with other modules to form sealed microfluidic interfaces. These interfaces may be press-fit or secured using mechanical tabs or pins. Modules call also be controlled and actuated by fluidic, electrical, magnetic, thermal, and mechanical interaction with an active control matrix lying beneath the module array plane. This control matrix can contain electrical connections, analog-digital interfaces, and magnetic and mechanical actuators, as well as thermocouples and fluidic connections, as well as outlet ports for liquid and particulate waste disposal from the microfluidic array. The control matrix and robotics can be connected to computers for software programmable real-time control.

Additionally, the manipulators can perform mechanical manipulation and operation of external equipment such as video cameras and third party external devices without human intervention.

The robotics system can be controlled by a programmable software package with an application programming interface that allows it to be used in conjunction with existing software programming languages such as C, C++, Visual Basic, Python, and MATLAB, as well as proprietary and open source robotics control software.

Monitoring of the robotic system can be performed using video monitoring, laser/optical sensors, as well as mechanical, temperature, and chemical sensors.

### IV. Operation

The methods of screening of the invention comprise using screening assays to identify, from a library of diverse molecules, one or more compounds having a desired activity. For example, modulating the amount of a target molecule. A “screening assay” is a selective assay designed to identify, isolate, and/or determine the structure of, compounds within a collection that have a preselected activity. By “identifying” it is meant that a compound having a desirable activity is isolated, its chemical structure is determined (including without limitation determining the nucleotide and amino acid sequences of nucleic acids and polypeptides, respectively) the structure of and, additionally or alternatively, purifying compounds having the screened activity). Biochemical and biological assays are designed to test for activity in a broad range of systems ranging from protein-protein interactions, enzyme catalysis, small molecule-protein binding, to cellular functions. Such assays include automated, semi-automated assays and high-throughput screening (HTS) assays.

In high-throughput screening (HTS) methods, many discrete compounds are preferably tested in parallel by robotic, automatic or semi-automatic methods so that large numbers of test compounds can be screened for a desired activity simultaneously or nearly simultaneously. It is possible to assay and screen up to about 6,000 to 20,000, and even up to about 100,000 to 1,000,000 different compounds a day using the integrated systems of the invention. Typically in HTS, target molecules are administered or cultured with whole animals or isolated cells, including the appropriate controls.

In one aspect of the invention, screening comprises contacting an animal with a library of compounds, some of which are modulators or ligands of the target or phenotype of interest, under conditions where complexes between the target and ligands can form, and identifying which members of the libraries are present in such complexes. In another non-limiting modality, screening comprises contacting an animal or a target enzyme with a library of compounds, some of which are inhibitors (or activators) of the target, under conditions where a product or a reactant of the reaction catalyzed by the enzyme produce a detectable signal. Thus, for example, inhibitors of target enzyme can decrease the signal from a detectable product or increase a signal from a detectable reactant, while activators of the target enzyme can increase the signal from a detectable product or decrease a signal from a detectable reactant.

The methods disclosed herein can be used for screening a plurality of test compounds. In certain embodiments, the plurality of test compounds comprises between 1 and 200,000 test compounds, between 1 and 100,000 test compounds, between 1 and 1,000 test compounds, between 1 and 100 test compounds, or between 1 and 10 test compounds. In certain embodiments, the test compounds are provided by compound libraries, whether commercially available or not, using combinatorial chemistry techniques. In certain embodiments, the compound libraries are immobilized on a solid support.

High throughput screening can be used to measure the effects of drugs on complex molecular events such as signal transduction pathways, as well as cell functions including, but not limited to, cell function, apoptosis, cell division, cell adhesion, locomotion, exocytosis, and cell-cell communication. Multicolor fluorescence permits multiple targets and cell processes to be assayed in a single screen. Cross-correlation of cellular responses will yield a wealth of information required for target validation and lead optimization.

In another aspect, the present invention provides a method for analyzing whole animals comprising providing an array of locations which contain multiple animals wherein the animals contain one or more fluorescent reporter molecules; scanning multiple animals in each of the locations to obtain fluorescent signals from the fluorescent reporter molecule in the animals; converting the fluorescent signals into digital data; and utilizing the digital data to determine the distribution, environment or activity of the fluorescent reporter molecule within the animals.

In another aspect, the present invention provides a method for analyzing whole animals comprising providing a location which contains an animal wherein the animal can optionally contain one or more reporter molecules; contacting the animal with a library of compounds; scanning the animal at multiple locations wherein each location contains a different detection system; obtaining detection signals and converting the signals into digital data; and utilizing the digital data to determine the distribution, environment or activity of the reporter molecules within the animals.

For example, the C. elegans or a zebra fish embryo can be placed in the chamber by a robotic handler. The animal can lie free in the fluid in the chamber, can be embedded in a low melting point agrose, or any other substance that can immobilize the animal but does not damage it or prevent gas/nutrient exchange. Each chamber will have a steady supply of defined buffer flowing through its microfluidic inlet channels. In addition, drugs can be administered to the animals in the chambers by either using the microfluidic channels or by robotic pipette handlers through a sliding lid, which may be plastic or glass, and can be retracted to expose the opening in the chamber for injection.

In some aspects of the invention, the microfluidic system described herein can comprise a lid that is configured to cover the openings of the chambers in the top surface of the substrate. The lid may be a sliding lid such that when the sliding of lid is at the open position, it provides an opening of the chambers for animals to be introduced prior to the start of the experiment, and/or to enable drugs to be introduced during the experiment. The lid can then be slid back into a covered position during the experiment. In another aspect of the invention, the lid can seal the chamber and enable efficient microfluidic flow in the chamber. The microfluidic flow can be under robotic control and can be used to maintain the animals by delivering the appropriate nutrients, removing waste, and for delivering test compound or library of compounds to the animals in the chambers.

In preferred embodiments, the assay provides a method of quantifying specific proteins in a biological sample, for example, a whole animal, or a cell.

In one aspect, the invention provides a method for screening candidate compounds for the treatment or prevention of a disease or disorder comprises contacting a whole animal with a candidate therapeutic agent and measuring the effects the compound has on the animal. The compound can then be further studied for any possible therapeutic effects. In certain preferred embodiments, the screening is conducted using high-throughput screening allowing for simultaneous diagnosing of many subjects at the same time.

In certain embodiments, the assay provides a method of diagnosing a disease or disorder comprising screening a biological sample from a patient in order to identifying and/or quantify a marker or molecule diagnostic of the particular disease or disorder. For example, a genetic marker, protein marker and the like. In another aspect, the invention provides a method of identifying subjects at risk of developing a disease or disorder comprising screening a biological sample from a patient and identifying and/or quantifying a marker or molecule diagnostic of the particular disease or disorder.

In one aspect, the invention provides a method for evaluating the effect of a compound on the behavior of the animals. The method provides administering a test compound to a mutant C. elegans as described above that is placed within a chamber of the microfluidics device of the invention, observing the behavior exhibited by the mutant C. elegans and comparing the observed behavior with that of a wild-type animal, wherein the observed behavior differences are associated with the test compound. The behavior to be observed can include forward and reverse locomotion and neck and nose movements of animals using video microscopy as they perform tasks such as foraging, chemotaxis, escape from noxious stimuli, mating, and social aggregation. As one of skill in the art will recognize, quantitative analysis can be performed on the measured behavioral parameters to detect changes in behavior induced by the compound. The growth of the animals, administering of the test compounds, observing the behavior of animals and comparing the differences in behavior can be performed using the robotic systems described above.

In one aspect, the invention provides a method for evaluating the effect of a compound on the neural activity of the animals. The method provides administering a test compound to a mutant C. elegans as described above, observing the neural activity exhibited by the mutant C. elegans and comparing the observed neural activity with that of a wild-type animal, wherein the differences in neural activity are associated with the test compound. The neural activity can be measured by using a genetically encoded indicator such as GCamp in one or more neurons to see how ensemble neural activity is affected by compounds. In another aspect, the neural activity can be measured in response to a behavior stimulus, such as, for example, starvation, hunger, or presentation of food, and the like.

In another aspect, the invention provides a method for evaluating the toxic effects or toxicity of a compound. The method provides administering a test compound to a mutant C. elegans as described above, observing the behavior exhibited by the mutant C. elegans and comparing the observed behavior with that of a wild-type animal, wherein the observed behavior differences are associated with the test compound. The behavior to be observed includes changes in movement of the wild-type and mutant C. elegans, changes in the rate of respiration, and changes in the lifespan of the wild-type and mutant C. elegans. As one of skill in the art will recognize, the methods and apparatus can be used to identify new molecular targets of a compound. For example, forward suppressor screens can be used to identify compounds with new molecular targets and mechanisms of action.

In yet another aspect, the invention provides an automated method for screening for compounds that can change muscle cell integrity, lipofuscin accumulation, fecundity, or respiration rate over an animal's lifetime and compounds that increase or decrease lifespan. Thus, for example, the methods of the invention provide administering a test compound to a mutant C. elegans that is placed within a chamber of the microfluidics device of the invention, observing the change in muscle cell integrity or lifespan exhibited by the mutant C. elegans and comparing with that of a wild-type animal, wherein the differences are associated with the test compound. The growth of the animals, administering of the test compounds, observing the animals and comparing the differences can be performed using the robotic systems described above. FIG. 4 provides representative data from growth assay in presence of drugs. Nemadipine (center boxes) is known to inhibit the growth of C. elegans by antagonizing egl-19, an L-type calcium channel. Verapamil (right boxes), a drug similar to nemadipine, also antagonizes egl-19 but is excluded from wild-type worms. ‘Druggable’ strains are permeable to verapamil.

While the invention has been particularly shown and described with reference to a preferred embodiment and various alternate embodiments, it will be understood by persons skilled in the relevant art that various changes in form and details can be made therein without departing from the spirit and scope of the invention. All printed patents and publications referred to in this application are hereby incorporated herein in their entirety by this reference.

What is claimed is:

1. An animal-based screening method to identify compounds, the method comprising:
providing an animal to one or more chambers in a microfluidics device module;
contacting the animal with a test compound; and
imaging the contents of the one or more chambers in order to obtain results in one or more chambers.

2. The method of claim 1, which is a high-throughput screening method.

3. The method of claim 1, where the animal is a whole animal, an embryo or a larvae.

4. The method of claim 3, wherein the whole animal is Caenorhabditis elegans (C. elegans).

5. The method of claim 4, wherein the C. elegans is wild-type or transgenic.

6. The method of claim 5, wherein the transgenic C. elegans comprises enhanced permeability to compounds.

7. The method of claim 5, wherein the transgenic C. elegans comprises tissue specific, location specific, developmental stage specific expression of human proteins, or combinations thereof.

8. The method of claim 5, wherein the C. elegans is sorted by complex object parametric analyzer and sorter or fluorescence activated cell sorter.

9. The method of claim 5, wherein the transgenic C. elegans comprises a reporter group.

10. The method of claim 9, wherein the reporter group is fluorescent reporter group or luminescent reporter group.

11. The method of claim 1, wherein imaging comprises fluorescence, luminescence, microscope, and combination thereof.

12. The method of claim 1, wherein the microfluidics device module comprises a plurality of detection zones.

13. The method of claim 12, wherein the plurality of detection zones comprises fluorescence or visual color change when exposed to a compound.

14. The method of claim 1, wherein the screening is performed robotically.
